(19) United States
(12) Patent Application Publication
(10) Pub. No.: US 2015/0158662 A1
(43) Pub. Date: Jun. 11, 2015

(54) Method and Apparatus for Flushing Eyes and Skin

(71) Applicant: Willam Kleyne, Grants Pass, OR (US)

(72) Inventor: Willam Kleyne, Grants Pass, OR (US)

(73) Assignee: Bio-Logic Aqua Technologies, Inc., Grants Pass, OR (US)

(21) Appl. No.: 14/608,553

(22) Filed: Jan. 29, 2015

Related U.S. Application Data
(62) Division of application No. 11/599,496, filed on Nov. 13, 2006.
(60) Provisional application No. 60/736,711, filed on Nov. 15, 2005.

(51) Int. Cl. B65D 83/62; A61F 9/00

(52) U.S. Cl. CPC B65D 83/62; A61F 9/00

(57) ABSTRACT

An apparatus and method for flushing or rinsing the skin and eyes by which an aqueous fluid consisting essentially of water is sprayed onto the skin and/or eyes for a time sufficient to remove contaminants on or in the skin or eyes.

This application is a divisional of pending U.S. patent application Ser. No. 11/599,496, filed on Nov. 13, 2006, which claims priority from U.S. provisional patent application Ser. No. 60/736,711, filed on Nov. 15, 2005.

### FIELD OF THE INVENTION

The present invention pertains to the field of the use of the topical administration of liquids to flush in wash the eyes in skin.

### BACKGROUND OF THE INVENTION

Presently available flushes for the eyes and for skin are based primarily on saline. Saline, although preferred for irrigating deep muscle wounds, is less satisfactory for flushing and rinsing superficial wounds or skin and/or eye contamination. Other methods for flushing the eye are based on rapid delivery of a stream or jet of water, such as from a tap, a hose, or a nozzle. These methods, although effective in flushing the eyes and skin and removing surface contaminants and foreign objects, are often irritating due to the mechanical effect of the stream or jet of water.

A significant need exists for a method of rinsing the eyes and skin that overcomes the disadvantages of the prior art.

### BRIEF DESCRIPTION OF THE DRAWINGS

FIG. 1 is a diagrammatic cut-away representation of a dual-compartment can apparatus of the invention.

### DESCRIPTION OF THE INVENTION

It has been discovered that flushing superficial wounds or skin and/or contamination is significantly improved by utilizing water lacking a significant level of inorganic or organic materials as a flush or rinse agent. It has been further discovered that such flushing is optimized when the water is administered as an aerosol spray rather than as a jet or stream. The tern “aerosol spray”, “spray”, and “aerosol” as used herein are synonymous and refer to a collection of liquid droplets finely dispersed in a gas, such as in air, so as to form a mist made up of discrete droplets having directional velocity. The term “aerosol” is less preferred than the term “spray” because the term “aerosol” has also come to mean a product in a pressurized can. The term aerosol is used herein in both contexts. When used in conjunction with the term “spray”, it is synonymous with that term. When used in conjunction with a product that is contained in and released from a container as a spray, it means the product itself.

As used herein, the terms “rinsing”, “flushing”, and “washing” are used interchangeably and refer to the use of a quantity of a liquid applied to the surface of an object in order to remove chemical, biological, and/or physical contaminants from on or in the object.

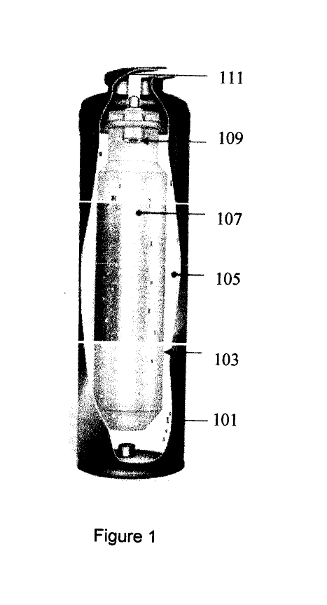

The invention, in one embodiment is an apparatus for rinsing the eyes or skin of a person in need thereof. According to this embodiment and as illustrated in a preferred embodiment in FIG. 1, the apparatus includes a rigid container 101, a flexible container 103 with the rigid container, a gas propellant 105 between the flexible and rigid container that is at a pressure higher than outside the apparatus, a fluid 107 consisting essentially of water within he flexible container, a valve 109 connected to the flexible container, and an actuator 111 connected to the valve which, when actuated, opens the valve and permits the fluid within the flexible container to exit the fluid container through the valve and to pass through the actuator to exit the apparatus in the form of a spray.

It is preferred that the fluid within the apparatus is sequestered so that external contaminants, especially biologic contaminants such as bacteria, viruses, and fungi, cannot enter the fluid and that the fluid remains sterile while in the apparatus.

In a preferred embodiment, the apparatus is in the form of a dual-compartment can, that is a can that maintains a propellant separate from a product. Examples of such dual-compartment cans which are well known in the art include “bag-in-can” and “hag-on-valve” cans. Such dual-compartment cans have many advantages. They maintain the sterility of fluid within the bag even if external conditions are unsanitary, permit the fluid in the bag to be sprayed from the can while holding the can at various angles, even upside-down, and provide for a consistency in spray from the apparatus, even when only as small amount of fluid remains in the bag following the spraying of a majority of the fluid that had originally been in the bag. Additional advantages of dual-compartment cans are that chemical propellants and preservatives are not needed and the fluid within the bag is insulated from extremes of heat and cold.

The rigid container that is suitable for the apparatus of the invention is made of a material that is gas and liquid impermeable and is preferably significantly inert to the gas that is contained inside the rigid container and to air outside the rigid container. Examples of suitable materials include plastics and metals, preferably coated metals such as painted metals.

The flexible container within the rigid container is a bag, preferably made of a plastic such as nylon or a flexible metal such as aluminum, like a flexible laminated aluminum bag or an aluminum foil coated plastic such as vinyl. The bag should be impervious to fluid and to gas and, therefore, should maintain a separation between fluid contained within the bag and the gas propellant that is exterior to the bag.

The gas propellant is any was that can he pressurized and inserted into a rigid container and act as a propellant to eject a liquid from a bag maintained with the rigid container. Preferably, the gas is inert to metal and to plastics and is not injurious to the environment. Suitable gas propellants include nitrogen, carbon dioxide, oxygen, and air. Nitrogen is especially preferred because of its abundance in the atmosphere and its relative non-reactivity.

The valve that is suitable for the apparatus of the invention is any valve that is capable of being open and closed and, when closed, prevents the fluid within the flexible container from exiting and, when opened, permits the fluid to exit the flexible container. Such valves are well known in the art.

The actuator that is suitable for the apparatus of the invention is any actuator that, when actuated, opens the valve and when not actuated, maintains the valve in a closed position. Such actuators are well known in the art. An example of a preferred actuator system is the ACC-U-SOL® Trigger Sprayer (Precision Valve Corporation, Yonkers, N.Y.).

Typically, the apparatus produces a spray in which the average droplet size is of as diameter between 20 and 1500 microns, ranging from as very fine aerosol mist to a coarse spray. Preferably, the average droplet diameter is (Dv(50)) between 50 and 500 microns, more preferably between 60 and 300 microns, and most preferably between 75 and 200 microns. In as most preferred embodiment, the average droplet size of the spray from the apparatus is about 100 microns, that is be seen 75 and 150 and even more preferably between 80 and 120 microns wherein individual droplets within the spray may range in diameter from about 4 microns or less to about 1000 microns or more.

The fluid within the flexible container consists essentially of water. The water that is suitable for the invention is essentially free of organic matter and has a low level of inorganic solutes. Preferably, the level of inorganic solutes in the water is less than 10% that of isotonic more preferably less than 5% that of isotonic saline, and most preferably less than 1% that of isotonic saline. In a specific preferred embodiment, the level of inorganic solutes in water is 1/140 that of isotonic saline. The pH of the water is between 5.0 and 8.5. Preferably, the water is at a neutral pH or lower, for example between 5.7 and 7.0. Most preferably, the pH is between 6.1 and 6.8. A most preferred pH is about 6.4.

In contrast to present apparatuses or devices for flushing the eyes, the apparatus of the present invention is a hand-held portable device that is capable of providing a flushing spray of a mist of droplets onto contaminated skin or eyes for an extended period of time. In a preferred embodiment, the apparatus will deliver a consistent flushing spray for a time greater than 16 seconds. In a more preferred embodiment, the apparatus will deliver as consistent flushing spray for 30 seconds or more. Most preferably, the apparatus of the invention will deliver a consistent flushing spray for 60 seconds or more, for example 90 seconds or more, or even 120 seconds or more. Because the fluid is delivered from the apparatus in the form of as mist, a relatively small volume of water may be contained within the apparatus and still provide such a long period of flushing time. For example, in a preferred embodiment, the quantity of fluid within the flexible container is between 2 and 10 ounces, such as 3 ounces or 7 ounces, thereby providing an apparatus having a weight which is comfortably held away from the body, with the arm partially or fully extended, for a time sufficient to flush contaminants from the skin or eyes.

The ability to provide such a long flushing, time and to provide such a flush time from such a small quantity of fluid are unexpected advantageous properties of the apparatus of the invention. Presently available non-mist flushing apparatuses, such as a 16 fluid oz. saline or water in the form of a stream or jet of fluid, provide a flushing spray that lasts no more than about 10 seconds. Such a brief time of flushing is often ineffective to remove surface contaminants from the skin or eyes. In contrast, the method and apparatus of the present invention provides at least 16 seconds, preferably at least 30 seconds, more preferably at least 60 seconds, even more preferably at least 90, and most preferably up to 120 seconds or more, of a forceful flushing spray that is effective to flush contaminants from the skin or the eye.

An additional unexpected advantageous property of the apparatus of the present invention is that the apparatus of the invention may he maintained at a temperature that is sufficiently low to cause the fluid in the apparatus to freeze. Such freezing does not deleteriously affect the apparatus of the invention as, following thawing of the fluid, the apparatus may be used as intended to flush skin or the eyes. This is because the gas, such as nitrogen, that is between the flexible container and the rigid container of the apparatus of the invention contracts at low temperatures and provides room for the water in the flexible container to expand, thus avoiding destruction of the apparatus due to the expanding frozen water. In contrast, presently available flushes that are not packaged in a dual-compartment can and that have undergone freezing must be discarded because the fluid of the flush liquid expands and damages the container, which compromises sterility.

The invention, in another embodiment, is a method for flushing the eyes or skin. According to this embodiment of the invention, a mist of droplets of an aqueous fluid consisting essentially of water is sprayed topically, such as into the eyes or skin, in an amount and for a time sufficient to remove chemical, biological, and/or physical contaminants from on or in the eyes or skin.

In a preferred embodiment, the mist of droplets is sprayed topically onto the eyes or skin from the apparatus of the invention as described above. According to the method of the invention, a person holds the apparatus of the invention, aims the exit of the actuator towards his or her eyes or skin, and actuates the actuator, thereby directing the sprayed mist towards his or her eyes or skin. Preferably, the apparatus is of a size that is able to be comfortably held in one hand with the arm partially or fully extended and the apparatus is thus held by an individual so that the tip of the actuator from which the aqueous spray will exit is situated between 8 and 24 inches from the contaminated site on the skin or eyes, more preferably between 8 and 18 inches, and most preferably between 8 and 12 inches.

Preferably, the person sprays the aqueous mist onto the eyes or skin for a time sufficient to flush any contaminants present. For example, although in many situations a very brief spray or 0.5 to 10 seconds is all that is needed to remove contaminants from the skin or eyes, in many situations, it is preferred that the person spray the aqueous mist from the apparatus of the invention for a time greater than 10 seconds, for example 16 seconds or more, or 30 seconds or more, or even 60, 90, or 120 seconds or more.

The method of the invention may be used to wash skin or a wound that has been contacted with irritating chemicals, such as gasoline, pepper spray, tear gas, or plant toxins such as from poison ivy or poison oak. The method of the invention may be used to flush skin or wounds that have been contaminated by physical contaminants such as dust, soil, or soot. The method of the invention may be used to flush eyes that are irritated by foreign particles, such as smoke, dust, or perspiration.

The invention, in all its embodiments, represents a significant advance in skin, wound, and eye care that is useful for emergency room physicians, nurses, and technicians, for shop, employer, or home first aid kits, and for use by policemen and firemen.

Further modifications, uses, and applications of the invention described herein will be apparent to those skilled in the art. It is intended that such modifications be encompassed in the following claims.

1. A hand-held apparatus for rinsing the eyes or skin of a person in need thereof comprising a rigid container, a flexible container with the rigid container, a gas propellant between the flexible and rigid container that is at a pressure higher than that of the atmosphere outside the apparatus, a fluid consisting essentially of water within the flexible container, a valve connected to the flexible container, an actuator connected to the valve which, when actuated, opens the valve and permits the fluid within the flexible container to exit the fluid container through the valve and to pass through the actuator to exit the apparatus m the form of an aerosol spray, wherein actuation of the valve provides a flushing flow of the aerosol spray fluid from the apparatus.

2. The hand-held apparatus of claim 1 which is a bag-in-can aerosol spray device.

3. The hand-held apparatus of claim 1 wherein the fluid contains inorganic solutes at a level that is less than 10% that of isotonic saline.

4. The hand-held apparatus of claim 3 wherein the level of inorganic solutes is less than 1% that of isotonic saline.

5. The hand-held apparatus of claim 1 wherein the aerosol spray is composed of discrete droplets having an average size of between 50 and 300 microns.

6. The hand-held apparatus of claim 1 wherein the actuation provides a flushing flow of the aerosol spray fluid from the apparatus for a sustained period of at least 16 seconds.

7. The hand-held apparatus of claim 6 wherein the actuation provides a flushing flow of the aerosol spray fluid from the apparatus for a sustained period of at least 30 seconds.

8. The hand-held apparatus of claim 7 wherein the actuation provides a flushing flow of the aerosol spray fluid from the apparatus for a sustained period of at least 60 seconds.

9. The hand-held apparatus of claim 8 wherein the actuation provides a flushing flow of the aerosol spray fluid from the apparatus for a sustained period of at least 90 seconds.

10. The hand-held apparatus of claim 1 Wherein the flexible container contains between 2 and 10 fluid ounces of the fluid.

11. The hand-held apparatus of claim 6 wherein the flexible container contains between 2 and 10 fluid ounces of the fluid.
